(19) United States
(12) Patent Application Publication
(10) Pub. No.: US 2003/0104017 A1
(43) Pub. Date: Jun. 5, 2003

(54) Epinastine formulation for oral administration

(75) Inventors: Koichi Wada, Kawabe-gun (JP); Manabu Nakatani, Kawanishi (JP); Toshimitsu Ohki, Ikeda (JP); Kenzo Toyoshima, Ikoma (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/269,315

(22) Filed: Oct. 11, 2002

Related U.S. Application Data
(60) Provisional application No. 60/342,997, filed on Dec. 20, 2001.

(30) Foreign Application Priority Data
Oct. 26, 2001 (DE) 101 52 973

(51) Int. Cl. A61K 31/55; A61K 9/00

(52) U.S. Cl. 424/400; 514/214.02

(57) ABSTRACT

A new formulation of epinastine in form of a powder (dry syrup) to be mixed with water prior to use is disclosed. The epinastine used can be either the free base or a pharmaceutically acceptable salt thereof.

The present invention refers to a new formulation of epinastine in the form of a powder to be mixed with water prior to use. Epinastine is useful in the treatment of allergies, pain (particularly chronic pain) and pain caused by inflammation, migraine, asthma, rhinitis, conjunctivitis and/or bronchitis (f.e. EP-B-0 035 749, EP 1000623). The formulation of the present invention is useful for treating allergies, dermatitis, rhinitis, conjunctivitis, bronchitis and asthma.

### STATE OF THE ART

Epinastine, chemically known as 3-amino-9,13b-dihydro-1H-dibenz-[c,f]imidazol[1,5-a]azepine, and its acid addition salts are disclosed in German Patent application P 30 08 944.2 which forms the basis for EP 0035749.

Chemically, epinastine is represented by the following formula:

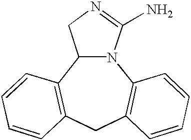

This formula does not reflect stereochemical properties.

Epinastine can be used either as free base or as a pharmaceutically acceptable salt thereof. Preferably, epinastine is used in the form of its hydrochloride salt.

If not specified further in the context of the present invention, the term epinastine is understood to be both the free base as well as pharmaceutically acceptable salts.

Methods for the preparation of epinastine are described in EP 0496306 or WO 01/40229.

Epinastine is marketed in Japan under the brand name Alesion®. In the Japanese market, it is most often used for its antihistaminic effects.

Currently, epinastine is available for use and is marketed in tablet format.

However, especially for children and elderly people, tablets are not always easy to take. Therefore, there is a need for a formulation that can be easily accessed by children and elderly people.

### SUMMARY OF THE PRESENT INVENTION

A suitable formulation for children and the elderly would be liquid formulations. However, epinastine has a strong taste of bitterness, which bitter taste is also long-lasting.

As a consequence, there was a need to develop either a neutral or good tasting liquid formulation of epinastine and/or one of its pharmaceutically acceptable acid addition salts.

Another objective of the present invention is to provide a liquid epinastine-formulation which can be stored for some time without adverse consequences to the stability of the formulation.

Still another objective of the present invention is to create an easy-to-handle epinastine formulation. A liquid formulation is also more desirably presented as a solution rather than a dispersion in order to improve its acceptance by the patient.

### DESCRIPTION OF THE PRESENT INVENTION

The present invention relates to a powder-like formulation (in essence, a dry syrup) of epinastine, either in enantiomeric, racemic form or a salt thereof, which formulation is mixed with water prior to use. It is essential that the powder dissolve in water very quickly.

In the context of the present invention, the term “powder” is used simultaneously with the term “dry syrup” which in turn stands for an essentially water-free admixture of the active form of epinastine, preferably epinastine-hydrochloride, and pharmaceutically acceptable additives and adjuvants necessary to form an aqueous, sweet-tasting formulation of the active substance when mixed with water.

Surprisingly, it was found that aqueous formulations of epinastine-hydrochloride result in two different bad tastes, both in the form of strong bitterness. On one hand, there is a quick-acting kind of bitterness, and on the other hand, there is a lasting bitterness that is tasted for quite a long time.

Unfortunately, the bitter taste of epinastine could not be masked by the use of one conventional taste-masking agent such as sucrose.

Instead, it was found that the taste of strong bitterness can be overcome by the combination of quick- and slow acting sweeteners and flavouring agents.

As a consequence thereof, a preferred embodiment of the formulation of the present invention should comprise at least one of each kind of these masking agents.

For the masking of quick-acting bitterness, sodium saccharin, erithritol and/or aspartame turned out to be effective. Another suitable group of compounds comprises sugars and sugar-derived polyols such as sucrose, D-sorbitol, glycerin and D-mannitol. Preferably, the formulation comprises sodium saccharin, erithritol and/or aspartame. Most preferred is the combination of sodium saccharin, erithritol and aspartame.

The amount of agent used to mask the quick-acting bitterness depends on agent used.

In case of sodium saccharin, it is between 0.1% w/w and 2.0% w/w of the powder formulation. Preferably the amount is 0.8.%

In case of erithritol, it is between 50% w/w and 95% w/w of the powder formulation. Preferably the amount is 75 to 80% w/w. Most preferred it is 80% w/w.

And in case of aspartame, it is between 1% w/w and 30% w/w of the powder formulation. Preferably the amount is 5 to 15% w/w. Most preferred it is 10%.

For masking lasting bitterness, glycyrrhizinates were found to be highly effective. Among them glycyrrhizinic acid and/or monoammonium glycyrrhizinate are the preferred ones. The most preferred one is monoammonium glycyrrhizinate.

The amount of monoammonium glycyrrhizinate in the powder formulation is 0.1% w/w and 3.0% w/w of the powder formulation. More preferred amounts are 0.1 to 1% w/w and the most preferred amount is 0.6%.

The formulation further may comprise adjuvants, among which are, for example, pH-adjusting agents. It is preferred to add such pH-adjusting agents to adjust the pH of the resulting liquid to a value of between about 5 and about 8, preferably 6 and 7. Among those agents are citric-acid, succinic acid, tartaric acid, acetic acid, citrates, acetates, vitamin C, hydrochloric acid, carbonates, phosphates, disodium phosphate, monosodium phosphate, sodium-, calcium-, potassium- and/or magnesium-hydroxide. Preferred are buffer substances like disodium phosphate.

Other additives often used in pharmaceutical formulation can also be added. Among these are

binding agents such as hydroxypropylcellulose, methylcellulose, hydroxypropylmethylcellulose, hydroxyethylcellulose, starch, dextrin, gelatine and polyvinylpyrrolidone, preferably hydroxypropylcellulose,

flow agents such as hydrated silicon dioxide, light anhydrous silicic acid,

odorizors such as sunfix orange No.22734 (commercial name, sunfix orange, which is preferred, contains orange flavour (30% w/w), acacia (30% w/w) and dextrin (40% w/w)), orange oil, mentha oil, eucalyptus, strawberry flavour, vanilla flavour, yoghurt flavour and other flavours known in the art,

colour pigments such as food yellow No.5, also being known as sunset yellow FCF (disodium salt of 6-hydroxyl-5-(4-sulfophenylazo)-2-naphthalenesulfonic acid), the latter being preferred, ferric oxide, and other food colour pigments, for example the ones known in Japan as food red No.3, 102, 105 and 106, food yellow No.4 and other food colours known in the art, and/or

preservatives such as benzoic acid, salts thereof, preferably sodium benzoate, paraoxybenzoic acids, salts thereof and other known preservatives, whereby sodium benzoate is preferred and

effervescent agents such as bicarbonate.

The amount of epinastine or its salt is between about 0.5% w/w and about 5% w/w of the powder formulation. More preferred is an amount of between 0.5% w/w and 2% w/w, and the most preferred amount is 1%.

The powder formulation preferably does not contain an active substance which is not epinastine or a pharmaceutically acceptable salt thereof.

The active substance, preferably epinastine-hydrochloride, and all the additives are mixed to a powder, and then the resulting powder is mixed with water to obtain a liquid formulation. Although the liquid formulation may either be a solution, suspension or a colloid, the preferred liquid formulation is a transparent and clear aqueous solution.

It is preferred to mix the powder formulation with water to obtain a liquid having a concentration of between about 250 mg per 5-50 ml and about 2000 mg per 10-100 ml, preferably the amount is between about 50 mg per 10 ml and about 2000 mg per 10 ml. If epinastine-hydrochloride is used, the amount in the context of the present invention is (approximately) the same for the free base.

In order to ensure that the patient can easily prepare the liquid formulation, the powder formulation (dry syrup) can be delivered in several packages, measured for ready use. In these packages, the water and the powder formulation are stored separately from each other. The package can further allow both components to mix in an easy way.

As a consequence thereof, the present invention also relates to a kit comprising two components, a) the inventive powder formulation and b) water, both components separated from each other.

Several bottles having special caps are known to solve or address this issue. Most often in such packages, the liquid solvent can be stored in a bottle of glass, plastic, metal and so on, while the cap for closing the bottle comprises a chamber to take the dry powder formulation. Prior to use, the patient can take the powder in the cap and mix it with water in the bottle. This mixing process can either be done consciously, meaning the patient actively takes the powder and puts it into the water. In other embodiments, the patient can initiate the mixing process in a more passive way, by, for example, just screwing, pressing, shaking the cap or the bottle in order to eliminate a barrier in the chamber containing the powder and by doing so allowing it to fall from the cap into the bottles.

Such packaging formats are disclosed, for example, in EP 0599189, EP 0344849, EP 0217425, EP 0093090, U.S. Pat. No. 3,802,604 and others. Herewith, all of these devices are incorporated by reference. Other, similar devices might be used, too. Besides such package forms, the dry syrup formulation can be stored in an aluminium or plastic-bag or in an aluminium or plastic bottle. The so stored powder then can be used with a pre-metered amount of water, stored in another package. Alternatively, the package can be freshly filled with drinking water at the time of use.

Other package systems may be employed as well.

In the context of the present invention the powder formulation can also be pressed into tablets to be dissolved in water, for example, an effervescent tablet. In such an embodiment, the tablet may additionally comprise an effervescent agent, such as bicarbonate.

Experimental
New dry and aqueous epinastine-Syrup-Formulation 0.5%.

| | Powder-Formulation No. | | | | | | |
|---|---|---|---|---|---|---|---|
| | 1 | 2 | 3 | 4 | 5 | 6 | 7 |
| epinastine | 5 | 5 | 5 | 5 | 5 | 5 | 5 |
| hydrochloroide [mg] | | | | | | | |
| glycerin [mg] | 0 | 0 | 0 | 0 | 0 | 0 | 350 |
| sucrose [mg] | 0 | 0 | 0 | 0 | 0 | 540 | 620 |
| anhydrous citric | 0 | 0 | 0 | 0 | 0 | 25 | 0 |
| acid [mg] | | | | | | | |
| monoammonium | 7 | 0 | 6 | 6 | 6 | 0 | 0 |
| glycyrrhizinate [mg] | | | | | | | |
| saccharin sodium [mg] | 0 | 6 | 4 | 0 | 5 | 5 | 0 |
| sodium fumarate [mg] | 10 | 10 | 0 | 10 | 5 | 0 | 10 |
| disodium | 0 | 0 | 0 | 3 | 15 | 10 | 0 |
| phosphate [mg] | | | | | | | |
| erithritol [mg] | 923 | 919 | 925 | 916 | 904 | 395 | 0 |
| aspartame [mg] | 60 | 60 | 60 | 60 | 60 | 25 | 0 |
| sodium benzoate [mg] | 0 | 0 | 0 | 0 | 0 | 0 | 15 |
| Results | | | | | | | |
| Appearance when | white | clear^{#} | white | clear^{#} | clear^{#} | clear^{#} | clear^{#} |
| mixed with water* | | | | | | | |
| Masking of | good | good | good | good | very | good | good |
| Bitterness | | | | | good | | |

*the amount of water is not significant. However, 1g of powder preferably should be readily soluble in 5-50 ml of water.
^{#}Clear in the meaning of transparent.

New dry and aqueous epinastine-Syrup-Formulation 1.0%

| | Powder-Formulation No. | | | | | | |
|---|---|---|---|---|---|---|---|
| | 8 | 9 | 10 | 11 | 12 | 13 | 14 |
| Epinastine hydrochloride | 10 | 10 | 10 | 10 | 10 | 10 | 10 |
| [mg] | | | | | | | |
| Glycerin [mg] | 0 | 0 | 0 | 0 | 0 | 0 | 350 |
| sucrose [mg] | 0 | 0 | 0 | 0 | 0 | 542 | 425 |
| anhydrous citric acid [mg] | 0 | 0 | 0 | 0 | 0 | 25 | 0 |
| monoammonium | 10 | 0 | 10 | 6 | 6 | 0 | 0 |
| glycyrrhizinate [mg] | | | | | | | |
| saccharin sodium [mg] | 0 | 10 | 5 | 0 | 8 | 0 | 0 |
| sodium fumarate [mg] | 20 | 10 | 0 | 15 | 5 | 0 | 10 |
| disodium phosphate [mg] | 0 | 0 | 0 | 10 | 15 | 0 | 0 |
| erithritol [mg] | 859 | 859 | 854 | 838 | 835 | 397 | 200 |
| aspartame[mg] | 100 | 100 | 100 | 100 | 100 | 25 | 0 |
| hydroxypropylcellulose | 0 | 20 | 20 | 20 | 20 | 0 | 0 |
| [mg] | | | | | | | |
| hydrated silicon dioxide | 1 | 1 | 1 | 1 | 1 | 1 | 0 |
| [mg] | | | | | | | |
| Odorizor* [mg] | 0 | (1) | (1) | (1) | (1) | (1) | 0 |
| food yellow No. 5** [mg] | 0 | (0.05) | (0.05) | (0.05) | (0.05) | (0.05) | 0 |
| sodium benzoate [mg] | 0 | 0 | 0 | 0 | 0 | 0 | 5 |
| Results | | | | | | | |
| Appearance when mixed | white | clear^{#} | white | clear^{#} | clear^{#} | clear^{#} | clear^{#} |
| with water*** | | | | | | | |
| Masking of Bitterness | good | good | good | good | very | good | good |
| | | | | | good | | |

*sunfix orange No. 22734 (commercial name) containing orange flavor (30% w/w), acacia (30% w/w) and dextrin (40% w/w).
** Another name of Food yellow no. 5 is sunset yellow FCF (disodium salt of 6-hydroxyl-5-(4-sulfophenylazo)-2-naphthalenesulfonic acid.
***the amount of water is not significant, however, 1 g of powder preferably should be readily soluble in 10-100 ml of water.
^{#}Clear in the meaning of transparent.

What is claimed is:

1. A pharmaceutical formulation comprising epinastine or an acid addition salt thereof, and at least two kinds of sweeteners or flavouring agents, wherein one of the at least two kinds of sweeteners or flavouring agents masks the quick-acting bitterness of epinastine or its salt and the other one masks the long-acting bitterness of epinastine or its salt.

2. The pharmaceutical formulation according to claim 1, wherein the sweetener or flavouring used to mask the quick-acting bitterness is selected from saccharin sodium, erithritol, aspartame and sugars or other polyols such as sucrose, glycerin, D-sorbitol and D-mannitol.

3. The pharmaceutical formulation according to claim 2, wherein the at least one sweetener or flavouring to mask the quick-acting bitterness is selected from saccharin sodium, erithritol and aspartame.

4. The pharmaceutical formulation according to claim 1, wherein the at least one sweetener or flavouring to mask the quick-acting bitterness is a combination of saccharin sodium, erithritol and aspartame.

5. The pharmaceutical formulation according to claim 1, wherein the at least one sweetener or flavouring to mask the long-acting bitterness is a glycyrrhizinate or glycyrrhinic acid.

6. The pharmaceutical formulation according to claim 5, wherein the glycyrrhizinate is mono-, di-ammonium glycyrrhizinate, di-, tri-sodium glycyrrhizinate or dipotassium glycyrrhizinate.

7. The pharmaceutical formulation according to claim 1, further comprising a pH-adjusting agent selected from the group of citric-acid, citrates, succinic acid, tartaric acid, acetic acid, acetates, vitamine C, hydrochloric acid, carbonates, sodium-, calcium-, potassium- and magnesium-hydroxide, and phosphates.

8. The pharmaceutical formulation according to claim 7, further comprising at least one adjuvant selected from the group of odorizers, colour pigments, preservatives, flow agents and binding agents.

9. A pharmaceutical formulation comprising epinastine-hydrochloride in an amount of between 0.5% w/w and 5%w/w, saccharin sodium in an amount of between 0.1% w/w and 2.0% w/w, erithritol in an amount of between 50% w/w and 95% w/w, aspartame in an amount of between 1% w/w and 30% w/w, and monoammonium glycyrrhizinate in an amount of 0.1% w/w and 3.0% w/w.

10. The pharmaceutical formulation according to claim 9, further comprising sodium fumarate, hydroxypropylcellulose, hydrated silicon dioxide, orange flavour, acacia, dextrin and disodium salt of 6-hydroxyl-5-(4-sulfophenylazo)-2-naphthalenesulfonic acid.

11. The pharmaceutical formulation according to claim 9, characterised in the formulation further comprises an effervescent agent.
